(19) United States
(12) Patent Application Publication
(10) Pub. No.: US 2008/0081797 A1
(43) Pub. Date: Apr. 3, 2008

(54) Topical formulations for the prevention of sexually transmitted disease and methods of producing the same

(76) Inventors: Craig Lichtblau, Jupiter, FL (US); Jose I. Iparraguirre, Miami, FL (US)

Correspondence Address: MCHALE & SLAVIN, P.A., 2855 PGA BLVD, PALM BEACH GARDENS, FL, 33410, US

(21) Appl. No.: 11/536,035

(22) Filed: Sep. 28, 2006

(51) Int. Cl. A61K 31/695

(52) U.S. Cl. 514/63; 424/401

(57) ABSTRACT

The present invention is directed towards various topical protective formulations which may be used as an adjunct in preventing the spread of a broad range of sexually transmitted diseases and the method by which it is produced. The product is intended to be used as a topical lotion, cream, emulsion, or the like. The use of a minimum of three film forming excipients in the following formulations have demonstrated unique skin protective barrier properties with enhanced persistence that inhibit transmission of sexually transmitted diseases.

### FIELD OF THE INVENTION

This invention relates to topical protective formulations for use in the prevention of the spread of sexually transmitted diseases (STDs); and particularly to unique topical formulations including a plurality of film forming excipients which together provide a barrier to help inhibit the transmission of STDs.

### BACKGROUND OF THE INVENTION

Despite years of research and education programs, the transmission of sexually transmitted diseases (STDs) remains a global health threat. STDs include bacterial types (e.g., gonorrhea, Chlamydia and syphilis) and viral types (human immunodeficiency virus (HIV), human papilloma virus (HPV) and hepatitis B). Although the communicability of these diseases varies, they are usually transmitted to the uninfected person through injured or exposed skin or mucous membranes during sexual contact. The continued emphasis on educating the population as to the use of “mechanical barriers” such as condoms has helped to decrease the morbidity of most STDs.

Chemical actives such as microbicides, antimicrobials, and spermicides, most notably, nonylphenoxypoly(ethyleneoxy)-ethanol (also referred to as nonoxynol-9) have been used in topical formulations to effectively reduce the rate of STD transmission, especially when used in conjunction with prophylactics. However, many of these chemical actives are harsh and have been shown to induce local irritation, inflammation, and ulcerations which might actually favor the transmission of STDs. Thus, a need exists for topical formulations that do not cause irritation and will help inhibit the spread of STDs, especially when used in combination with condoms, and thereby provide an additional degree of protection from contamination, should the condom become damaged.

### PRIOR ART

Although there are numerous patents and publications directed to formulations containing chemical actives, such as, microbicides, antimicrobials, spermicides, and drug delivery carriers (liposomes, micelles), none of the known prior art teach formulations comprising non-irritating agents that provide a physical barrier to the permeation of pathogens.

U.S. Patent Application 2003/0143189 A1 to Askill et al., is directed to a method of treating skin lesions by forming a polymeric film over the lesions to inhibit proliferation of infectious agents in the lesions. These compositions also include one or more chemical agents in combination therewith.

U.S. Pat. No. 6,835,717 to Hildreth discloses a method of reducing the risk of a sexually transmitted pathogen by contacting a pathogen within the composition which consists of β-cyclodextrin.

U.S. Pat. No. 6,821,958 to Hershline discloses a method of preventing viral transmission using an alkylsulfate derivative of sulfated dextrin as a topical formulation.

U.S. Pat. No. 6,582,711 to Asmus et al., discloses an antimicrobial hydroalcoholic composition having a cationic polymeric thickener.

U.S. Pat. No. 6,355,235 to Cone et al., consists of an antibody capable of trapping sperm and a pharmaceutical carrier.

U.S. Pat. No. 6,328,991 to Myhling discloses a chemical composition to prevent the transmission of sexually transmitted diseases comprising nonylphenoxpoly-(ethyleneoxy)-ethanol, benzalkonium chloride and povidone iodine.

U.S. Pat. No. 5,439,685 to Augros is a composition of an agent active against microorganisms responsible for sexually transmitted diseases together with a film forming agent such as dimethylpolysilozane, and benzalkonium chloride as a spermicidal.

U.S. Pat. No. 4,952,411 to Fox, Jr. et al., is a composition of silver sulfadiazine, alone or in combination with cholorhexidine or sodium deoxycholate (antimicrobial and detergent).

### SUMMARY OF THE INVENTION

The purpose of this invention is to provide non-irritating protective formulations to be used as an adjunct in preventing the spread of a broad range of sexually transmitted diseases and the methods of producing the same. The products of the present invention can be formulated as a topical lotion, cream, solution, emulsion, or the like. The use of at least three film forming excipients in the following formulations have demonstrated unique skin protective barrier properties with enhanced persistence that inhibit “skin to skin” and “sore to sore” transmission of pathogens (e.g., viruses, bacteria, fungi, parasites, ectoparasites and mycoplasmas) linked to communicable diseases.

Skin protectant products are regulated under CFR 21 Part 347 “Skin Protectant Drug Products for Over the Counter Human Use”. The official description of a skin protectant is “a drug product that temporarily protects injured or exposed skin or mucous membrane surfaces from harmful or annoying stimuli, and may help provide relief to such surfaces.” These regulations cover applicable ingredients, as well as labeling requirements for over the counter skin protectants. Active ingredients officially classified as skin protectants compositions as well as certain combinations of these compositions are listed in the CFR 21 Sec. 347.10, reproduced in Table 1 below. In accordance with the instant invention, the phrase “a skin protectant composition provided in a skin protecting effective concentration” will be understood to mean any of the following ingredients at their designated concentration:

TABLE 1
These include any of the following within the concentration
range specified:

| Ingredient | Concentration |
|---|---|
| Allantoin | 0.5 to 2.0% |
| Aluminum hydroxide gel | 0.15 to 5.0% |
| Calamine | 1.0 to 25.0% |
| Cocoa Butter | 50.0 to 100.0% |
| Cod liver oil | 5.0 to 13.56%(in accordance with Sec. |
| | 347.20(a)(1) or (a)(2), provided the |
| | product is labeled so that the quantity |
| | used in a 24-hr period does not exceed |
| | 10,000 USP Units vitamin A and 400 USP |
| | Units cholecalciferol. |
| Colloidal oatmeal | 0.007 minimum; 0.003 minimum in |
| | combination with mineral oil |
| | in accordance with Sec. |
| | 347.20(a)(4). |
| Dimethicone | 1.0 to 30.0% |
| Glycerin | 20.0 to 45.0% |
| Hard fat | 50.0 to 100.0% |
| Kaolin | 4.0 to 20.0% |
| Lanolin | 12.5 to 50.0% |
| Mineral oil | 50.0 to 100.0%; 30.0 to 35.0% in |
| | combination with colloidal oatmeal in |
| | accordance with Sec. 347.20(a)(4). |
| Petrolatum | 30.0 to 100.0% |
| Topical starch | 10.0 to 98.0% |
| White petrolatum | 30.0 to 100.0% |
| Zinc acetate | 0.1 to 2.0% |
| Zinc carbonate | 0.2 to 2.0% |
| Zinc oxide | 1.0 to 25.0% |

It has been discovered that incorporation of at least one of the aforementioned skin protectants compositions listed in Table 1 in combination with other film forming excipients, in particular, a film-forming emollient (e.g., Isostearyl Isostearate) and silicone-containing excipient (e.g., SILKYWAX, a product of Dow Corning) in a formulation for topical application which results in a product that temporarily protects injured or exposed skin or mucous membrane surfaces from harmful or annoying stimuli, thereby preventing cross-contamination of pathogenic microorganisms responsible for sexually transmitted diseases (e.g., Herpes simplex virus type 1 and type 2 (HSV-1, HSV-2), human immunodeficiency virus (HIV), or the like) through skin or mucous membrane surfaces. The novel formulation products of this invention are persistent in that they form a film or barrier on healthy or even damaged skin.

The hydrophobic portions of the instant formulations utilize a combination of at least three film forming excipients: skin protectant(s) listed in Table 1, silicones and silicone derivatives or like equivalents, and a film-forming emollient. These three ingredients are dispersed by the emulsifier throughout the continuous aqueous phase. They melt and spread over the skin by body heat. This forms a physical hydrophobic layer which resides on the skin surface (including mucosal membranes) and provides a barrier which would inhibit penetration of liquids and pathogens which are primarily hydrophilic in nature. When used in combination with consistent and careful use of condoms, the products of the present invention help inhibit the spread of STDs and protect the skin from contamination should the condom become damaged.

Accordingly, it is an objective of the instant invention to teach various topical protective formulations to be used in preventing the spread of a broad range of sexually transmitted diseases.

It is a further objective of the instant invention to teach a method for producing protective formulations wherein adherence to particular process parameters results in a unique final product.

Another objective of the present invention is to teach formulations which are condom compatible, meaning that they are latex friendly and provide effective lubricity.

It is yet another objective of the instant invention to teach skin protective formulations wherein contact with the skin results in the formation of a hydrophobic skin protective surface layer.

Yet another objective of the invention is to teach products formulated as a lotion, cream, solution, emulsion, or other topically applied product.

Other objects and advantages of this invention will become apparent from the following description, wherein are set forth, by way of illustration and example, certain embodiments of this invention.

### DETAILED DESCRIPTION OF THE INVENTION

Detailed embodiments of the instant invention are disclosed herein, however, it is to be understood that the disclosed embodiments are merely exemplary of the invention, which may be embodied in various forms. Therefore, specific functional and structural details disclosed herein are not to be interpreted as limiting, but merely as a basis for the claims and as a representation basis for teaching one skilled in the art to variously employ the present invention in virtually any appropriately detailed structure.

Glycerin (INCI name: Glycerin) is a non-irritating humectant and film former. It is also water soluble. Moreover, glycerin is compatible with latex products and provides extended lubricity, which makes it a common base for many products designed for genital use. While glycerin is the preferred skin protectant composition of the present invention, other suitable skin protectant compositions and their amounts effective to provide skin protection as listed in Table 1 above could be used herein (e.g., Dimethicone).

TEFOSE 63 is a PEG (polyethylene glycol) palmito-stearate, non-ionic base for O/W (oil-in-water) formulations (a product of Gattefosse Corp., Hawthorne, N.Y., USA); INCI name: PEG-6 stearate/glycol stearate/PEG-32. Although the use of TEFOSE 63 is preferred, it is contemplated that any other non-irritating, non-ionic emulsifier could be used without departing from the scope of the present invention.

The phase “silicone-containing excipient” refers to any silicone or silicone derivatives suitable for cosmetic and pharmaceutical applications which acts as an emollient and forms a water-repelling film, including silicone gums. According to one non-limiting embodiment, SILKY WAX is the preferred. SILKY WAX is a semi-occlusive, low melting (about 53 degrees C) silicone wax which acts as an emollient and water-repelling film former (a product of Dow Corning); INCI name: steroxytrimethylsilane/stearyl alcohol.

The phase “film-forming emollient” refers to any excipient suitable for cosmetic and pharmaceutical applications which forms a water-repelling film. According to a preferred, albeit non-limiting embodiment, the film-forming emollient is Isostearyl isostearate. Isostearyl isostearate is a double branched ester (oil soluble) and emollient with film formation properties; INCI name: isostearyl isostearate. Although isostearyl isostearate is preferred other emollients can be used herein, for example, LUTROL OP 2000 (a product of BASF Aktiengeselllschaft) INCI NAME: polyoxypropylene (26) oleate.

EMERESSENCE is a natural preservative (INCI name: phenoxyethanol; a product of Cognis Care, Inc., Cincinnati, Ohio, USA) and may be used in any of the instant formulations to prevent microbial growth. Any other preservative suitable for cosmetic and pharmaceutical use that does not produce skin irritation while providing optimal asepsis could be used herein, for example, BIOPEIN (a product of BIO-BOTANICA, Inc., Hauppauge, N.Y., USA), PARAGON MEPB (a mixture of Methyl, Ethyl, Propyl, and Butyl Parabenzene; a product of McIntyre Group, Ltd., University Park, Ill., USA) or a like equivalent or combination thereof.

Excipients useful in forming the skin protective formulations (lotions, solutions, creams, emulsions, or the like) according to the present invention are described in the following non-limiting example.

### EXAMPLE 1

In formulating a batch of the skin protective cream according to one preferred embodiment of the invention, excipients useful in the manufacture of this product were added in the following approximate amounts:

| | |
|---|---|
| EXCIPIENT | WT/WT % RANGES |

Water phase

| | |
|---|---|
| (1) DEIONIZED WATER | Q.S. |
| (2) GLYCERIN | 20.0–45.0 |

Oil Phase

| | |
|---|---|
| (3) TEFOSE 63 | 3.0–20.0 |
| (4) Isostearyl Isostearate | 2.0–10.0 |
| (5) SILKY WAX | 2.0–10.0 |
| (6) *EMERESSENCE | 0.1–1.0 |

*optional excipient.

The following method steps were followed:

Step (A) The Water Phase is formulated by combining the appropriate amounts of following ingredients into a container having at least one heating means and mixer:

The mixer is engaged at rate sufficiently rigorous to yield a homogeneous water phase while heating to about 75 degree C.

The Oil Phase excipients listed below are weighed and added to another container with a heating means and mixer:

Step (B) All of the aforementioned Oil Phase ingredients are stirred while being heated to approximately 75 degrees C until completely blended.

Step (C) If desired the EMERESSENCE (or like preservative) is added to the Oil Phase immediately prior to adding the Water phase into the Oil phase.

Step (D) The Water Phase is then added to the Oil Phase while stirring slowly; slow planetary or axial stirring should be sufficient.

Step (F) The mixture is slowly cooled slowly to room temperature (approximately 25 degrees C) while stirring.

While not wishing to be bound to any particular theory, it is believed that these three film forming excipients (e.g., Isostearyl Isostearate, Glycerin, and SILKY WAX) form a neutral and hydrophobic layer barrier which is also believed to interact with the skin thereby having a stabilizing effect upon the hydrophobic layer, which results in the enhanced persistence of the product. The use of a silicone-containing excipient, e.g. SILKY WAX, or a like equivalent, acts in-coordination with at least one of the skin protectants compositions as defined in CFR 21 Sec. 347.10 (Table 1) and the film-forming emollient, e.g., Isostearyl Isostearate, and melts when contacted with the heat of the body. This in turn forms a physical hydrophobic layer that provides a barrier which appears to inhibit penetration of liquids and sexually transmitted pathogens (viral, bacterial) which are primarily hydrophilic in nature. This property helps protect the user from contamination due to sexual contact with infected individuals.

Other ingredients which may be useful in the manufacture of the product may be included, for example, LABRAFIL M 1944 CS (INCI: Oleoyl macrogolglycerides, produced by Gattefosse Corp) can be added to the oil phase formulation of Example 1 as a co-emulsifier. LABRIFIL M 1944 SC is an unsaturated polyglycolized glyceride obtained by partial alcoholysis of apricot kernel oil, consisting of glycerides, and polyethylene glycol esters- and amphiphilic oil, solvent and/or emulsifier.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and drawings/figures.

All patents and publications mentioned in this specification are indicative of the levels of those skilled in the art to which the invention pertains. All patents and publications are herein incorporated by reference to the same extent as if each individual publication was specifically and individually indicated to be incorporated by reference. It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein. The excipients and related compounds, methods, procedures, and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. An oil-in-water formulation for the prevention of sexually transmitted diseases, characterized by enhanced skin protective properties comprising in combination:
(1) a skin protectant composition provided in a skin protecting effective concentration;
(2) non-ionic emulsifier from about 3.0 to about 20.0 wt/wt %;
(3) a silicone-containing excipient, in the range from about 2.0 to about 10.0 wt/wt %;
(4) a film-forming emollient in the range from about 2.0 to about 10.0 wt/wt %; and
(5) water;
wherein contact with the skin results in the in situ formation of a skin protective barrier layer effective in preventing transmission of sexually transmitted diseases.

2. The formulation of claim 1, wherein said emulsifier is a mixture of polyethyleneglycol-6 stearate, glycol stearate and polyethyleneglycol-32 stearate.

3. The formulation of claim 1 wherein said skin protectant is glycerin.

4. The formulation of claim 1 wherein said skin protectant is dimethicone.

5. The formulation of claim 1 further including a co-emulsifier, in the range of from about 0.10 to about 0.5 wt/wt %.

6. The formulation of claim 5, wherein said co-emulsifier is an oleoyl macrogolglyceride.

7. The formulation of claim 1, further including at least one anti-microbial preservative agent in the range of about 0.1 to about 1.0 wt/wt %.

8. The formulation of claim 7, wherein said at least one anti-microbial preservative includes a mixture of methyl-,ethyl-,propyl- and butyl-parabenzene.

9. The formulation of claim 7 wherein said at least one anti-microbial preservative includes phenoxyethanol.

10. The formulation of claim 1 wherein said silicone-containing excipient is a mixture of stearoxytrimethylsilane and stearyl alcohol.

11. The formulation of claim 1 wherein said film-forming emollient is isostearyl isostearate.

12. The formulation of claim 1, wherein said film-forming emollient is polyoxypropylene (26) oleate.

13. The formulation of claim 1, wherein said formulation is in the form of a cream.

14. The formulation of claim 1, wherein said formulation is in the form of a lotion.

15. An oil-in-water cream for the prevention of sexually transmitted diseases, characterized by enhanced skin protective properties comprising in combination:
(1) a mixture of polyethyleneglycol-6 stearate, glycol stearate and polyethyleneglycol-32 stearate; at about 12.0 wt/wt %;
(2) isostearyl isostearate; at about 8.0 wt/wt %;
(3) a mixture of stearoxytrimethylsilane and stearyl alcohol; at about 5.0 wt/wt %;
(4) glycerin; at about 20.0 wt/wt %;
(5) phenoxyethanol; at about 0.5 wt/wt %; and
(6) QS with deionized water;
wherein contact with the skin results in the in situ formation of a skin protective barrier layer.

16. An oil-in-water cream for the prevention of sexually transmitted diseases produced by a method comprising the steps of:
1) forming homogeneous water phase mixture by mixing a sufficient quantity of deionized water, and about 20.0 wt/wt % of glycerin within an appropriately sized vessel;
2) weighing in the amounts stated, about 12.0 wt/wt % of a mixture of polyethyleneglycol-6 stearate, glycol stearate and polyethyleneglycol-32 stearate, about 8.0 wt/wt % of isostearyl isostearate, and about 5.0 wt/wt % of a mixture of stearoxytrimethylsilane and stearyl alcohol into an appropriately sized container while mixing to form a homogeneous oil phase and heating to within a temperature range of about 70° C. to about 85° C.;
3) add about 0.5 wt/wt % of phenoxyethanol to said oil phase; and
4) mixing said water phase and said oil phase together while cooling to room temperature to yield a homogeneously blended cream formulation.

17. An oil-in-water formulation for the prevention of sexually transmitted diseases produced by a method comprising the steps of:
1) forming homogeneous water phase mixture by mixing a sufficient quantity of water and about 20.0 to about 45.0 wt/wt % of a skin protectant composition within an appropriately sized vessel;
2) weighing in the following amounts, about 3.0 to about 20.0 wt/wt % of an non-ionic emulsifier, about 2.0 to about 10 wt/wt % of a film-forming emollient, and about 2.0 to about 10.0 wt/wt % of a silicone-containing excipient into an appropriately sized container while mixing to form a homogeneous oil phase and heating to within a temperature range of about 70° C. to about 85° C.; and
3) mixing said water phase and said oil phase together while cooling to room temperature to yield a homogeneously blended cream formulation.

18. The formulation of claim 17, further including adding at least one anti-microbial preservative agent in the range of about 0.1 to about 1.0 wt/wt % prior to step 3).

19. The formulation of claim 18, wherein said at least one anti-microbial preservative includes a mixture of methyl, ethyl, propyl and butyl parabenzene.

20. The formulation of claim 18, wherein said at least one anti-microbial preservative includes phenoxyethanol.

21. The formulation of claim 17, wherein said emulsifier is a mixture of polyethyleneglycol-6 stearate, glycol stearate and polyethyleneglycol-32 stearate.

22. The formulation of claim 17 wherein said skin protectant is glycerin.

23. The formulation of claim 17, wherein said skin protectant is dimethicone.

24. The formulation of claim 17 wherein, said silicone-containing excipient is a mixture of stearoxytrimethylsilane and stearyl alcohol.

25. The formulation of claim 17 wherein, said film-forming emollient is isostearyl isostearate.

26. The formulation of claim 17, wherein said film-forming emollient is polyoxypropylene (26) oleate.
